(19) United States
(12) Patent Application Publication
(10) Pub. No.: US 2019/0290178 A1
(43) Pub. Date: Sep. 26, 2019

(54) ADAPTIVE ELECTRONIC PROMOTION OF OPTIMAL HEALTH BEHAVIOR

(71) Applicant: International Business Machines Corporation, Armonk, NY (US)

(72) Inventors: Paul R. Bastide, Boxford, MA (US); Jonathan Dunne, Dungarvan (IE); Liam S. Harpur, Skerries (IE); Robert Loredo, North Miami Beach, FL (US)

(21) Appl. No.: 15/934,217

(22) Filed: Mar. 23, 2018

(51) Int. Cl. A61B 5/16; G16H 50/30; G16H 10/60; G16H 10/20

(52) U.S. Cl. CPC A61B 5/165; G16H 10/20; G16H 10/60; G16H 50/30

(57) ABSTRACT

Methods and systems are provided to identify an individual's health risk of developing a disease or disorder, and to motivate the user to adopt a behavioral change to mitigate the progression of the identified disease or disorder. An individual's medical history is analyzed to determine diseases that a user is at risk of developing, as well as positive and negative behaviors of the user relating to the disease. For a negative behavior, the system identifies a behavioral change that is to be promoted to the individual through a cohort. The system then sends notifications to the cohort, for the cohort to promote the behavioral change to the individual via a client system associated with the individual. Methods and systems are also provided to send notifications to a cohort to promote maintaining the behavioral change to reduce the likelihood that the individual will revert to former behavior.

### BACKGROUND

### 1. Technical Field

Present invention embodiments relate to adaptive systems and methods to promote optimal health behaviors, and more specifically, to analyzing an individual's medical history to identify health risks, mapping health risks to positive behaviors to mitigate the health risks, and electronically promoting the positive behaviors through a cohort in an adaptive manner.

### 2. Discussion of the Related Art

Health problems and medical issues are a significant source of economic drain, resulting in about 70 million workers reporting missed days due to illness each year. In aggregate, these absences may reduce economic output by up to $260 billion per year.

Early detection of medical diseases has been proven to improve employee health and enhance workplace productivity. As the Centers for Disease Control recognizes, preventive health care improves health outcomes. Patients who become aware of a disease or disorder in its early stages may take action to reduce or halt the progression of the disease. For instance, patients who are diagnosed with prediabetes, a condition associated with impaired glucose tolerance, typically progress to developing type II diabetes. However, behavioral changes with regard to diet and activity level may mitigate or even halt the progression of this disease. Successful individuals may also reduce or prevent the occurrence of complications arising from type II diabetes including cardiac disease, kidney disease, and neurological conditions.

Although physician offices, community outreach programs, companies and other organizations promote a wide range of events to promote access, offering free and/or on-site health screenings, etc., many individuals still fail to utilize such services due to underestimating their health risk, lack of adequate planning, etc. While health screenings have been shown to act as a catalyst for changing behaviors and improving health outcomes for individuals, motivating an individual to attend such health screenings is a challenge, especially if individuals believe that they are not at significant risk for health problems. Additionally, maintaining a behavioral change is difficult, as individuals frequently relapse to former patterns of behavior.

### SUMMARY

According to embodiments of the invention, methods, systems and computer readable media, in a data processing system, are provided to adaptively provide electronic notifications to promote a behavioral change of a user. A medical history of a user is analyzed to identify a risk of developing a disease. A behavior of the user that promotes the development of the disease is determined. The behavior is mapped to a behavioral change, wherein the behavioral change mitigates the development of the disease. The behavioral change is promoted to the user through a cohort, by providing electronic notifications to the cohort. The electronic notifications provided to the cohort are adaptively modified until the user adopts the behavioral change.

It is to be understood that the Summary is not intended to identify key or essential features of embodiments of the present disclosure, nor is it intended to be used to limit the scope of the present disclosure. Other features of the present disclosure will become easily comprehensible through the description below.

### BRIEF DESCRIPTION OF THE DRAWINGS

Generally, like reference numerals in the various figures are utilized to designate like components. Through the more detailed description of some embodiments of the present disclosure in the accompanying drawings, the above and other features and advantages of the present disclosure will become more apparent.

FIG. 1 is a block diagram of an example computing environment for identifying a behavioral change and adaptively promoting the behavioral change through a cohort in accordance with embodiments of the present disclosure.

FIG. 2 is a flowchart of an example computer-implemented method of analyzing an individual's medical history to identify behavioral changes in accordance with embodiments of the present disclosure.

FIG. 3 is a flowchart of an example computer-implemented method of adaptively determining a cohort in accordance with embodiments of the present disclosure.

FIG. 4 is a flowchart of an example computer-implemented method of adaptively modifying notifications until a behavioral change occurs in accordance with embodiments of the present disclosure.

FIG. 5 is a flowchart of an example computer-implemented method of identifying a behavioral change to mitigate a health risk for a user, and adaptively promoting the behavioral change to the user through a cohort, in accordance with embodiments of the present disclosure.

### DETAILED DESCRIPTION

Methods and systems are provided to identify a health risk of developing a disease or disorder, to motivate users to adopt a behavioral change to mitigate the progression of the identified disease or disorder, and to maintain the behavioral change to reduce the likelihood of the development or progression of the disease or disorder.

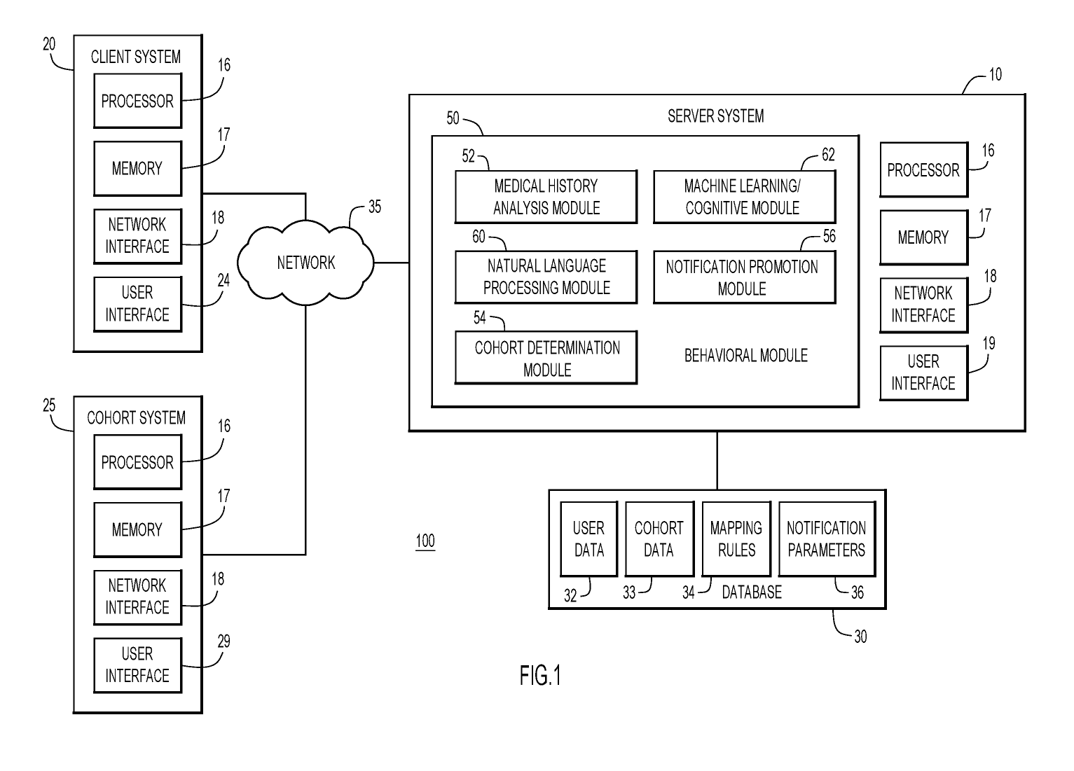

An example computing environment for use with present invention embodiments is illustrated in FIG. 1. Specifically, the environment includes one (or more) server system 10, one or more client or end-user systems 20, and one or more cohort systems 25. Although the example computing environment in FIG. 1 shows a single server system 10, a single client system 20, and a single cohort system 25, it is understood that this configuration may be extended to any number of server systems 10, to any number of client systems 20, and to any number of cohort systems 25. Server system 10, client systems 20, and cohort systems 25 may be remote from each other and may communicate over a network 35. The network may be implemented by any number of any suitable communications media (e.g., wide area network (WAN), local area network (LAN), Internet, Intranet, etc.). Alternatively, server system 10, client systems 20, and cohort systems 25 may be local to each other, and may communicate via any appropriate local communication medium (e.g., local area network (LAN), hardwire, wireless link, Intranet, etc.). Server system 10 may provide notifications to both client systems 20 and cohort systems 25. Cohort systems 25 may promote notifications to client systems 20. Activity of client systems 20 may be monitored by server system 10 to assess whether a behavioral change has occurred.

Client systems 20 enable users to receive notifications from server system 10 as well as promotion of such notifications by cohort systems 25, wherein the notifications comprise information pertaining to a positive behavioral change (e.g., upcoming health screenings, dietary guidelines to improve health, upcoming physical activities, or any other information pertaining to a positive health behavior). Client systems 20 may include a desktop, laptop, tablet, smart phone, wearable device, or any other device used for electronic communication, and notifications may be provided or promoted to any suitable device. The notifications from server system 10 comprise information to promote a behavioral change in order to improve an individual's health. The notification may be directly provided to client systems 20 and/or to cohort systems 25, wherein members of the cohort promote the notification to client systems 20 within a given timeframe.

The server system 10 may include behavioral module 50, which includes a plurality of submodules (e.g., medical history analysis module 52, cohort determination module 54, notification promotion module 56 as well as natural language processor 60 and machine learning/cognitive module 62, etc.) to identify positive behaviors and promote such behaviors to client systems through a cohort. The server may also comprise user interface 19, e.g., for configuration of the behavioral module 50.

A database system 30 may store various types of information for the analysis (e.g., user data 32 including information about at-risk diseases, desired behavior changes, etc.; cohort data 33 comprising identification of a cohort, general rules governing composition of a cohort, etc.; mapping rules 34 including mapping of negative behaviors to (positive) behavioral changes; notification parameters 36 governing characteristics of notifications, which may also include optimized parameters derived from longitudinal analysis of a population. The database system 30 may be implemented by any conventional or other database or storage unit, may be local to or remote from server system 10, client systems 20, and cohort systems 25 and may communicate via any appropriate communication medium (e.g., local area network (LAN), wide area network (WAN), Internet, hardwire, wireless link, Intranet, etc.).

The client systems 20 may present a graphical user (e.g., GUI, etc.) or other user interface 24 (e.g., command line prompts, menu screens, etc.) to display information from notifications to a user pertaining to a behavioral change, may provide reports to the user of resultant health changes arising from behavioral changes (e.g., improvements in BMI, improved fitness metrics, improved blood work results, etc.), and may communicate with server system 10 to monitor changes in individual behavior.

Cohort systems 25 may also present a graphical user (e.g., GUI, etc.) or other user interface 29 (e.g., command line prompts, menu screens, etc.) to receive notifications from server system 10 and may allow cohort members to promote such notifications to client systems 20. Cohort systems 25 may also provide an indication to server system 10 when a notification has been promoted to client systems 20. In cases where an indication to server system 10 has not been provided, server system 10 may send periodic reminder notifications to cohort systems 25 until receiving confirmation that promotion of the notification has occurred. Promotion of the notification may occur electronically (e.g., via an email message, an instant message, an alert from an application, an alert from a wearable device, etc.) or non-electronically (e.g., a verbal communication, etc.).

Server system 10, client systems 20, and cohort systems 25 may be implemented by any conventional or other computer systems preferably equipped with a display or monitor, a base (e.g., including at least one processor 16, one or more memories 17 and/or internal or external network interfaces or communications devices 18 (e.g., modem, network cards, etc.)), optional input devices (e.g., a keyboard, mouse or other input device), and any commercially available and custom software (e.g., server/communications software, module, browser/interface software, etc.).

Alternatively, one or more client systems 20 may include the functionality of behavioral module 50 when operating as a stand-alone unit, such that longitudinal analysis is limited to the client system and other client systems that have been configured to share information with the client system. In a stand-alone mode of operation, the client system stores or has access to the data (e.g., user data 32, cohort data 33, mapping rules 34, and notification parameters 36, etc.), and includes behavioral module 50 to identify behavioral changes for improved health. Client system may generate and send notifications to cohort systems 25. The graphical user (e.g., GUI, etc.) or other interface (e.g., command line prompts, menu screens, etc.) solicits information from a corresponding user to which the behavioral change is being promoted and monitors activity of the user, and may provide reports regarding health status changes to the user.

Behavioral module 50 may include one or more modules or units to perform the various functions of present invention embodiments described herein. The various modules (e.g., medical history analysis module 52, cohort determination module 54, notification promotion module 56 as well as natural language processor 60 and machine learning/cognitive module 60) may be implemented by any combination of any quantity of software and/or hardware modules or units, and may reside within memory 17 of the server and/or client systems for execution by processor 16.

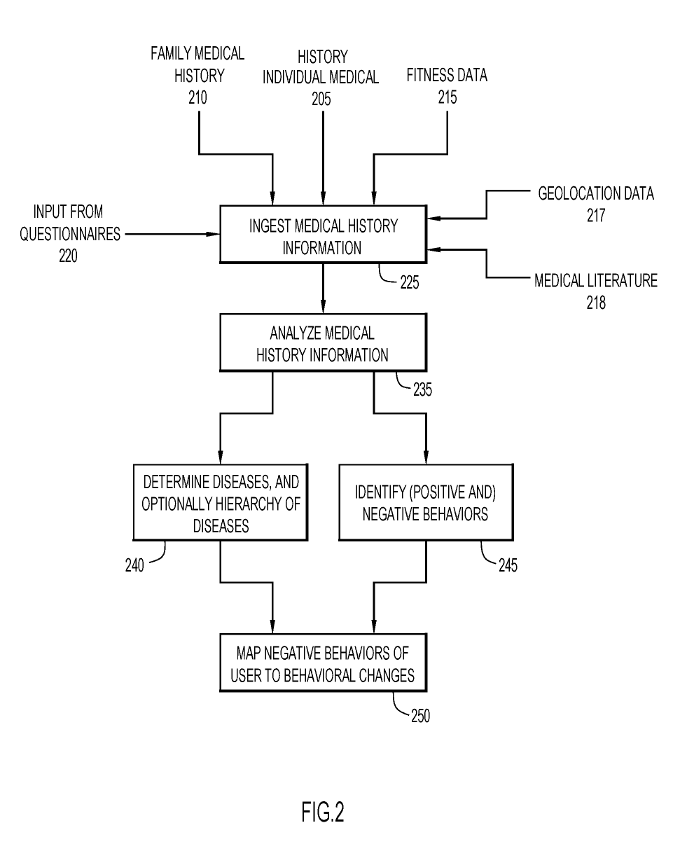

FIG. 2 is a flowchart depicting example operations of medical history analysis module 52. This module identifies diseases or disorders that an individual may have or is at risk for developing, identifies negative behaviors that may be contributing to the progression of the disease, and maps the identified negative behaviors to (positive) behavioral changes to mitigate the progression or development of the disease, which are then promoted to the user via client systems 20 by cohort systems 25 (or directly).

Medical history information may comprise a variety of data sources, including but not limited to an individual's medical history 205, a family medical history 210, fitness data 215 from wearable devices or fitness applications, geolocation data 217, medical literature 218, and input from questionnaires 220. Any one or more of these data sources may be provided to the system for ingestion and analysis to determine at risk diseases or disorders as well as positive and negative behaviors corresponding to the mitigation or progression of the disease or disorder, respectfully.

Medical history 205 may include an individual's family history of diseases (e.g., identification of relatives with heart disease, cancer, etc. and their relationship to the individual), age, ethnicity, previous medical screenings (e.g., weight, height, BMI, blood analysis (e.g., blood glucose, cholesterol levels, liver enzymes, CBCs, cancer or inflammatory marker screenings, other genetic or metabolic tests, etc.), heart/pulse rate, systolic and diastolic blood pressure, etc.), previous surgeries, previous medical conditions and treatments, prescribed medications, other diagnostic and imaging tests (e.g., chest x-rays, EKGs, EEGs, etc.), physician notes, or any other form of medical information that may be present in a patient's medical record. Physical activity level and dietary information may also be included as part of individual's medical history, e.g., as part of physician comments. In some aspects, the medical history may be obtained from commercial electronic medical or health records systems or custom development solutions for electronic medical records.

In some embodiments, the system may also consider related family medical history 210. The system may be configured to include any level of family history determined appropriate to assess risk for a particular disease. For example, the system may limit medical information to the individual's immediate family (e.g., mother, father, brothers, sisters, children, etc.). In other embodiments, the system may be configured to retrieve family history and electronic health records for extended family members (e.g., grandparents, aunts, uncles, cousins, etc.) or distant family (e.g., second cousins, third cousins, etc.). In other cases, the system may be configured to limit family history to genetic relationships or within a similarity threshold, e.g., a family member having a similar health profile as the individual.

In other aspects, the system may include fitness data 215 obtained from wearable electronic devices and fitness applications that track an individual's level of activity, heart rate, dietary habits, etc.

In still other aspects, geolocation data 217 may be included as a risk factor for developing particular diseases. For example, individuals living in regions with high levels of inactivity or who are at risk for developing particular types of disease based on their location (e.g., cardiovascular disease, diabetes, cancer, etc.) may be automatically targeted for particular behavioral interventions based on their location.

Medical literature 218 may include any source of electronic medical information such as peer reviewed medical journals, medical databases, governmental or global health organizations, physician-based medical organizations, etc. The system may analyze such information to identify recommended positive behaviors, e.g., based on a consensus of the medical community for preventing or mitigating particular diseases or disorders.

In still other aspects, input from questionnaires 220 are included to identify risk of developing particular diseases for an individual. The questionnaire may be provided to family members or other individuals who have knowledge of an individual's current behavior (e.g., diet, exercise habits, stress levels, sleeping habits, etc.) to help assess negative and/or positive behaviors of the individual relative to a particular disease or disorder.

Medical history information (205-220) may be ingested, at operation 225, into the system from any suitable source and may comprise both unstructured and structured data. For information in the form of free or unstructured text, e.g., physician comments, scanned images subjected to optical character recognition, etc., language normalizing software may be utilized to standardize the free text, e.g., SnoMed, Carecom, etc., relative to medical terminology. In some aspects, the system may automatically correct common typographical errors and perform other standardization operations to reduce the likelihood of omission of relevant information. In other aspects, comments may be specifically scanned for disease indicators, e.g., keywords such as diabetes, results of blood glucose levels, etc.

At operation 235, medical history information may be analyzed, using natural language processing systems 60 and/or machine learning systems 62, to analyze medical history information 205-220 to identify diseases that an individual is at risk of developing, and once these diseases have been identified, to identify behaviors which may mitigate (positive behaviors) or exacerbate (negative behaviors) the development or progression of the disease. For instance, particular diseases may have genetic and environmental risk factors which increase an individual's risk of developing a disease or disorder. An individual may have a high risk of developing diabetes if the individual has multiple risk factors associated with the development of diabetes, such as a family history of diabetes, a sedentary lifestyle, impaired blood glucose levels or fasting blood glucose levels that exceed a predetermined threshold, increases or decreases in other biomarkers relative to a predetermined threshold that are prognostic of diabetes, genetic mutations associated with the development of diabetes, presence of other diseases that may contribute to the development and progression of diabetes, etc. As another example, an individual may have a high risk of developing heart disease if the individual has multiple risk factors associated with the development of heart disease, such as a family history of heart disease, a sedentary lifestyle, high cholesterol levels that exceed a predetermined threshold, increases or decreases in biomarkers relative to a predetermined threshold that correlate with the progression and development of heart disease, genetic mutations associated with the development of heart disease, presence of other diseases that contribute to the progression of heart disease, etc. Behavioral changes such as changes in diet, increasing physical activity, etc., may mitigate the progression of these diseases.

In some aspects, the system may evaluate data from past health screenings to assess whether a disease is progressing or is being successfully managed. For example, if past health screenings are reviewed, the system may determine whether BMI has increased or decreased, whether fasting blood glucose levels have increased or decreased, etc.

Medical history analysis module 52 may also analyze patient records in aggregate, as well as information provided in the medical literature, to predict whether an individual is at risk for developing a particular disease. For example, the system may identify novel combinations of risk factors (e.g., particular biomarkers in combination with other factors, such as physical factors) associated with the presence of a particular disease, and may select behavioral changes accordingly.

At operation 240, the system may determine diseases, and optionally, a hierarchy of diseases, such that diseases (parent diseases) that cause or contribute to the development of other diseases (child diseases) are higher in the hierarchy. For example, diabetes is often associated with a variety of complications including cardiovascular diseases, kidney disorders, neurological disorders, retinopathy, Alzheimer's disease, etc. Accordingly, for an individual with multiple disorders falling within this group, the system may determine to direct intervention, at least initially, to the parent disease rather than considering each ailment separately.

Machine learning and/or natural language processing modules 60, 62 may be used to generate the disease hierarchy. For example, natural language processing techniques may be used to analyze repositories (including medical literature 218) to extract medical information and machine learning or other cognitive techniques may be used to identify novel correlations/connections of diseases, e.g., to identify new correlations between diseases, in some cases, in a hierarchical manner. In some aspects, to generate a disease hierarchy, information from medical literature 218 (including scientific databases, peer reviewed medical journals, etc.) may be used.

At operation 245, the system may determine positive or negative behaviors based on the individual's medical history for a particular disease that an individual is at risk of developing. Positive behaviors may mitigate the progression of the disease, while negative behaviors may advance progression of the disease. For example, for prediabetes, positive behaviors that mitigate the progression to diabetes include but are not limited to reducing carbohydrate intake, increasing level of activity, consistent monitoring of blood glucose levels and maintenance of said levels below a threshold, achieving a healthy body mass to weight ratio, etc. Negative behaviors that exacerbate the development of diabetes include but are not limited to maintaining a high carbohydrate intake, maintaining a sedentary lifestyle, failing to adequately monitor blood glucose levels, etc.

Other examples of negative behaviors include but are not limited to out-of-date medical screening information, e.g., not having a preventive health care screening in a designated time frame.

Positive and negative behaviors may be relative to a specific disease or disorder. For example, an individual with sleep apnea may have a different set of positive or negative behaviors than an individual with prediabetes. Accordingly, the system may create a set of positive and negative behaviors for each respective disease or disorder relative to the patient.

In other cases, information or rules regarding determining positive behaviors may be provided by one or more medical professional organizations (e.g., a physician, a group of physicians, or an organization representing groups of physicians), wherein positive behaviors reflect current medical recommendations to mitigate specific diseases.

In some cases, the system may evaluate a user's overall behavior. For users engaging in both positive and negative behaviors relative to a particular disease, the system may determine that further behavioral change would lead to incremental results. For example, a prediabetic user who regularly exercises, follows a low-carbohydrate diet, and has maintained their respective BMI, may receive a small benefit from a behavioral change associated with glucose monitoring 4x a day, and therefore, the system may focus on another disease.

At operation 250, for negative behaviors (wherein a negative behavior can be an absence of a positive behavior), the system maps the negative behavior to a specific behavioral change (e.g., a positive behavior) needed to mitigate the progression of the disease. For example, a behavioral change for not attending a past preventive health screening would be to attend an upcoming preventive health screening. A behavioral change for a sedentary lifestyle would be to increase the level of activity, e.g., engaging in regular exercise. Once the positive behavior is identified, a cohort is selected to promote (provide ongoing communication to the user targeted for a behavioral change) and to maintain the behavioral change.

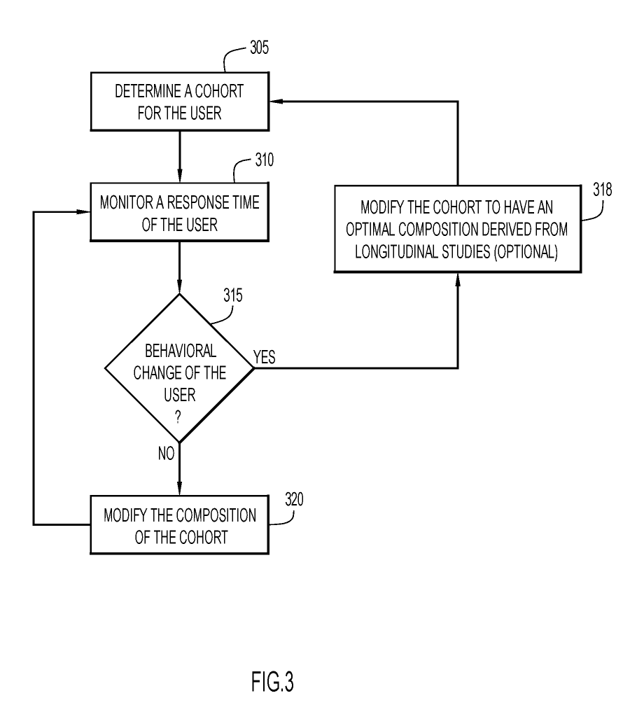

FIG. 3 is a flowchart depicting example operations of cohort determination module 54. At operation 305, a cohort is generated to promote behavioral changes with respect to the user. In some aspects, cohort determination module 54 scans the person's social network, email connections, social exercise/health application connections, and other electronic information including text messages to identify contacts of the user. The cohort determination module 54 may generate a cluster or cohort comprising contacts, e.g., high-frequency contacts, of the user to promote the behavioral change. In some embodiments, contacts are analyzed to determine whether they already engage in the behavioral change or any positive health behavior, and those who do are given priority for inclusion in the cohort over contacts who do not. In other aspects, members of the cohort may be analyzed to determine success rates for promoting a behavioral change, and those who have higher success rates for promoting the behavioral change are given priority over those who have been less successful.

At operation 310, electronic data associated with the user is monitored to determine whether the behavioral change has been adopted. Electronic data may include text messages, email, information from wearable fitness devices or fitness applications, geographic location (e.g., spending more time at a park), etc. At operation 315, the module determines based on the user response whether the behavioral change has been adopted. If the change has been adopted, the cohort may not be adjusted, or may be optionally adjusted as part of optimization recommendations from longitudinal studies as shown in operation 318.

If the behavioral change has not been adopted, the cohort may be modified at operation 320 to include new members of the cohort. For example, members of the cohort that fail to encourage a positive behavior or have low success rate of promoting the behavioral change may be removed, while members that encourage positive behaviors and have high success rates are maintained. New members may also be added to the cohort.

As discussed, cohorts may be dynamically selected by the system as a function of time and/or based upon longitudinal studies, according to operations 318. As the behavioral system 50 collects and analyses parameters and their corresponding values involving successful behavioral changes, the system can determine the size and composition of the cohort (e.g., number of members, and relationship of members to the individual), and optionally may adjust the composition of the cohort as a function of time. In some embodiments, optimal cohorts may be different for initiating the behavioral change and for maintaining the change.

As an example, initially, the cohort may be selected among family and close friends. This first cohort composition may be implemented to maximize the likelihood of successfully causing a behavioral change (e.g., close friends and immediate family). Once the behavioral change has been adopted, a second cohort composition may be implemented, wherein the composition is implemented to maximize the likelihood of maintaining the behavioral change (e.g., family, friends with healthy behaviors, social media groups or organizations engaging in healthy behaviors, etc.).

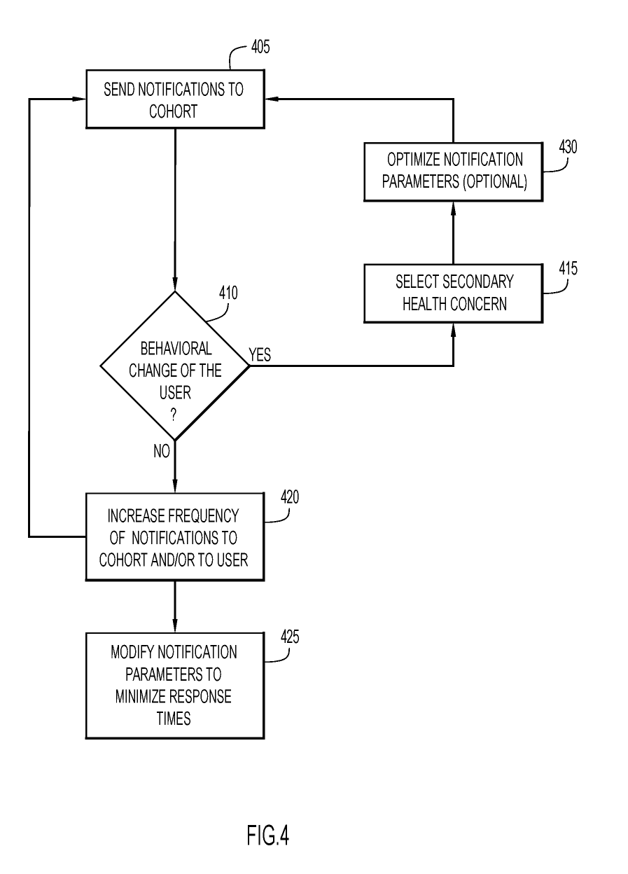

FIG. 4 is a flowchart depicting example operations of notification promotion module 58. The notification promotion module 58 sends to the cohort, wherein the cohort is tailored to the user, notifications comprising the positive behavioral change at operation 405. In some cases, the module may provide the notification to the cohort as a message or template that may be sent to the user, wherein the message/template is specifically tailored to promote a behavioral change as determined by the medical history analysis module 52. In some cases, the notification to the cohort may be sent via email, instant messaging, telephone, or any other form of electronic communication. In other embodiments, the notifications may appear as a percentage of advertisements sent to the user. Reminder notifications may be sent to the cohort on a periodic basis, as determined by a notification frequency parameter, to promote the behavioral changes on an ongoing basis. The cohort may promote the notification to client systems 20 via email, instant messaging, telephone, or any other form of electronic communication.

At operation 410, the notification promotion module 58 determines if the behavioral change has occurred. If the behavioral change has occurred, the system may generate a different notification to promote another behavioral change to address a secondary health concern at operation 415. For example, as the health of the user begins to improve, the system may address lower priority health concerns. For example, once a patient's behavior has been modified to slow or prevent the progression of prediabetes to type II diabetes, the system may focus on secondary health issues, such as arthritis, joint pain, etc.

User response data may be provided to the notification promotion module as a function of time. When the user engages in a positive behavior (e.g., attending a preventive health screening, exercising, achieve a reduction in fasting blood glucose, etc.), the system may analyze a variety of notification parameters, e.g., the types of notifications, frequency of notifications, times of notifications, source of notifications (e.g., family, friend, employer, online social group, applications, entity, etc.) to identify optimal parameter values to efficiently modify an individual's behavior. For example, the system may determine an optimal number of reminders, an optimal number of cohort members (e.g., individuals, applications, entities, etc.) from which to receive reminders, in order to successfully promote a behavioral change. For example, the system may determine that eight promotions of a behavioral change from the cohort results in positive behavioral change for an individual.

If the user is determined to have not adopted the behavioral change, at operation 420, the notification promotion module may increase the frequency of notifications to the cohort or send notifications directly to the user via client systems 20. At operation 425, the notification promotion module may modify notification parameters in order to find a set of parameters that lead to a response from the user. If successful, the modified parameters may be provided to the database, as part of a longitudinal study, to determine optimal parameters across a population to which a behavior change is being promoted.

At operation 430, as additional data is collected from a population of users, e.g., as part of a longitudinal study, the notification parameters leading to a behavioral change may be provided to a database to determine optimal parameter values for the population. For example, an optimal time to send notifications to the cohort may be a time in the morning, prior to the start of the workday. An optimal time to promote the behavior to the user may be during a lunch break, e.g., a time when appointments may be made, etc. As another example, the system may determine an optimal frequency of delivering notifications for the behavioral change, e.g., and may determine that two “healthy” notifications per month are optimal.

In some cases, the cohort determination module and notification promotion module may interface with fitness applications to improve derivation of targeted health plans (e.g., Couch-to-5 k, Runtastic, etc.) In some cases, the system may obtain information pertaining to local health events, and may use this information to send additional notifications to the cohort and/or user via client systems 20.

As the system collects and analyzes data from a population, the system may adjust the composition of the notification. For example, the notification promotion module may modify the content of the notifications based on other notifications that have successfully promoted the behavioral change.

Example notifications include but are not limited to promotion of a health event (e.g., a preventive health screening, a physical activity such as a 5K, etc.), an event having a health component (e.g., a primary event such as festival or concert having a secondary medical screening, etc.), or an event pertaining to a friend or cohort (e.g., a positive behavioral changes such as completing a 5K, etc.).

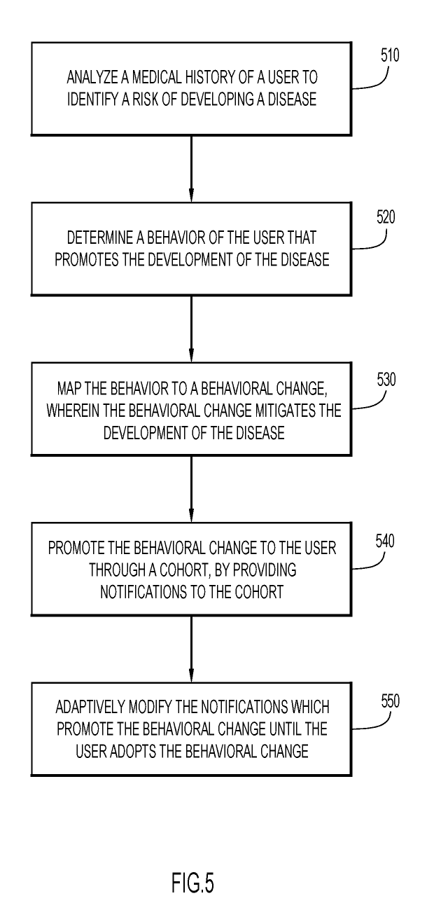

FIG. 5 is a flowchart of an example computer-implemented method of identifying a behavioral change to mitigate a health risk for a user, and adaptively promoting the behavioral change to the user through a cohort, in accordance with embodiments of the present disclosure. At operation 510, a medical history of a user is analyzed to identify a risk of developing a disease. At operation 520, a behavior of the user that promotes the development of the disease is determined. At operation 530, the behavior is mapped to a behavioral change, wherein the behavioral change mitigates the progression or development of the disease. At operation 540, the behavioral change is promoted to the user through a cohort, by providing notifications to the cohort. At operation 550, adaptively modify the notifications which promote the behavioral change until the user adopts the behavioral change.

Advantages of the present techniques include the ability to effectively and dynamically target specific behavioral changes to mitigate health concerns or risks. By including a variety of resources, e.g., social media, exercise and fitness applications, wearable health technology, cohorts, etc., the user may receive notifications from a variety of sources to maximize the likelihood that a behavioral change will occur. If the user is not responsive to the notifications, the system will dynamically adapt, e.g., by increasing the frequency of notifications, by altering the cohort, by changing the content of the notifications, etc.

As more user data becomes available, the system may use machine learning and NLP techniques to identify optimal notification parameter values. Accordingly, the parameter values provided to an initial group may be different from the parameter values provided to a later group of users. Thus, the system may learn, as a function of time, parameter values that are optimal, leading to smaller or reduced response times from users, wherein a response time is the time from which a behavioral change is promoted to the time at which the behavioral change occurs. Thus, the system may engage in continual/ongoing analysis of data, and may dynamically adjust and refine parameter values, thereby improving the response time across the population on an ongoing basis.

As new types of medical screening technologies are available, the system may provide notifications regarding advanced diagnostics, e.g., new type of imaging studies, new biomarkers, etc. The system may also adjust the type and frequency of screenings as a person ages (e.g., recommending cancer screenings as a person ages).

Present methods and techniques may be applied to any situation in which a behavior modification of a user is desired, e.g., changes in financial behavior (e.g., to reduce personal spending), changes in a workplace environment (e.g., to increase productivity, to increase skill level, to reduce stress, etc.), changes in an academic environment (e.g., to study more effectively, to improve grades, etc.), and so forth.

It will be appreciated that the embodiments described above and illustrated in the drawings represent only a few of the many ways of implementing embodiments for modifying a user's behavior by promoting the behavioral change through a cohort.

The environment of the present invention embodiments may include any number of computer or other processing systems (e.g., client or end-user systems, server systems, cohort systems, etc.) and databases or other repositories arranged in any desired fashion, where the present invention embodiments may be applied to any desired type of computing environment (e.g., cloud computing, client-server, network computing, mainframe, stand-alone systems, etc.). The computer or other processing systems employed by the present invention embodiments may be implemented by any number of any personal or other type of computer or processing system (e.g., desktop, laptop, PDA, mobile devices, etc.), and may include any commercially available operating system and any combination of commercially available and custom software (e.g., behavioral module 50, cohort software, client software, server software, etc.). These systems may include any types of monitors and input devices (e.g., keyboard, mouse, voice recognition, etc.) to enter and/or view information.

It is to be understood that the software (e.g., behavioral module 50, medical history analysis module 52, cohort determination module 54, notification promotion module 56, natural language processing module 60, machine learning/cognitive module 62, etc.) of the present invention embodiments may be implemented in any desired computer language and could be developed by one of ordinary skill in the computer arts based on the functional descriptions contained in the specification and flow charts illustrated in the drawings. Further, any references herein of software performing various functions generally refer to computer systems or processors performing those functions under software control. The computer systems of the present invention embodiments may alternatively be implemented by any type of hardware and/or other processing circuitry.

The various functions of the computer or other processing systems may be distributed in any manner among any number of software and/or hardware modules or units, processing or computer systems and/or circuitry, where the computer or processing systems may be disposed locally or remotely of each other and communicate via any suitable communications medium (e.g., LAN, WAN, Intranet, Internet, hardwire, modem connection, wireless, etc.). For example, the functions of the present invention embodiments may be distributed in any manner among the various end-user/client and server systems, cohort systems, and/or any other intermediary processing devices. The software and/or algorithms described above and illustrated in the flow charts may be modified in any manner that accomplishes the functions described herein. In addition, the functions in the flow charts or description may be performed in any order that accomplishes a desired operation.

The software of the present invention embodiments (e.g., behavioral module 50, medical history analysis module 52, cohort determination module 54, notification promotion module 56, natural language processing module 60, machine learning/cognitive module 62, etc.) may be available on a non-transitory computer useable medium (e.g., magnetic or optical mediums, magneto-optic mediums, floppy diskettes, CD-ROM, DVD, memory devices, etc.) of a stationary or portable program product apparatus or device for use with stand-alone systems or systems connected by a network or other communications medium.

The communication network may be implemented by any number of any type of communications network (e.g., LAN, WAN, Internet, Intranet, VPN, etc.). The computer or other processing systems of the present invention embodiments may include any conventional or other communications devices to communicate over the network via any conventional or other protocols. The computer or other processing systems may utilize any type of connection (e.g., wired, wireless, etc.) for access to the network. Local communication media may be implemented by any suitable communication media (e.g., local area network (LAN), hardwire, wireless link, Intranet, etc.).

The system may employ any number of any conventional or other databases, data stores or storage structures (e.g., files, databases, data structures, data or other repositories, etc.) to store information (e.g., user data 32, cohort data 33, mapping rules 34, notification parameters 36, etc.). The database system may be implemented by any number of any conventional or other databases, data stores or storage structures (e.g., files, databases, data structures, data or other repositories, etc.) to store information (e.g., user data 32, cohort data 33, mapping rules 34, notification parameters 36, etc.). The database system may be included within or coupled to the server and/or client systems. The database systems and/or storage structures may be remote from or local to the computer or other processing systems, and may store any desired data (e.g., user data 32, cohort data 33, mapping rules 34, notification parameters 36, etc.).

The present invention embodiments may employ any number of any type of user interface (e.g., Graphical User Interface (GUI), command-line, prompt, etc.) for obtaining or providing information (e.g., user data 32, cohort data 33, mapping rules 34, notification parameters 36, etc.), where the interface may include any information arranged in any fashion. The interface may include any number of any types of input or actuation mechanisms (e.g., buttons, icons, fields, boxes, links, etc.) disposed at any locations to enter/display information and initiate desired actions via any suitable input devices (e.g., mouse, keyboard, etc.). The interface screens may include any suitable actuators (e.g., links, tabs, etc.) to navigate between the screens in any fashion.

The present invention embodiments are not limited to the specific tasks or algorithms described above, but may be utilized for modifying a user's behavior by promoting the behavioral change through a cohort.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises”, “comprising”, “includes”, “including”, “has”, “have”, “having”, “with” and the like, when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

What is claimed is:

1. A method, in a data processing system comprising at least one processor and at least one memory, the at least one memory comprising instructions executed by the at least one processor to cause the at least one processor to adaptively provide electronic notifications to promote a behavioral change of a user, the method comprising:
analyzing a medical history of a user to identify a risk of developing a disease;
determining a behavior of the user that promotes the development of the disease;
mapping the behavior to a behavioral change, wherein the behavioral change mitigates the development of the disease;
promoting the behavioral change to the user through a cohort, by providing electronic notifications to the cohort; and
adaptively modifying the electronic notifications provided to the cohort until the user adopts the behavioral change.

2. The method of claim 1, wherein the medical history includes one or more of a family history, a user's medical history, fitness data, medical literature, geolocation data, and input from questionnaires.

3. The method of claim 1, further comprising:
identifying a plurality of diseases that the user is at risk of developing or has been diagnosed with;
generating a hierarchy of the diseases to identify a parent disease that contributes to one or more child diseases; and
selecting the behavioral change to mitigate the development of the parent disease.

4. The method of claim 1, further comprising:
analyzing, for a population of users, characteristics of electronic notifications provided to corresponding cohorts that successfully promoted a behavioral change in order to determine optimal characteristics of the electronic notifications; and
dynamically adapting characteristics of the electronic notifications provided to the cohort to the optimal characteristics in order to achieve a reduced response time of the user with regard to adopting the behavioral change.

5. The method of claim 1, further comprising:
analyzing, for a population of users, a composition of corresponding cohorts for the population of users that successfully promoted a behavioral change in order to determine an optimal cohort composition; and
dynamically adapting a composition of the cohort to the optimal cohort composition in order to achieve a reduced response time of the user with regard to adopting the behavioral change.

6. The method of claim 1, further comprising:
determining that the behavioral change has not occurred within a specified timeframe; and
increasing a frequency of notifications to the cohort until the behavioral change occurs.

7. The method of claim 1, further comprising:
determining that the behavioral change has not occurred within a specified timeframe; and
modifying the cohort by adding members that have been successful with promoting behavioral changes and/or removing members that have not been successful with promoting behavioral changes.

8. A system for adaptively promoting a behavioral change, the system comprising at least one processor configured to:
analyze a medical history of a user to identify a risk of developing a disease;
determine a behavior of the user that promotes the development of the disease;
map the behavior to a behavioral change, wherein the behavioral change mitigates the development of the disease;
promote the behavioral change to the user through a cohort, by providing electronic notifications to the cohort; and
adaptively modify the electronic notifications provided to the cohort until the user adopts the behavioral change.

9. The system of claim 8, wherein the medical history includes one or more of a family history, a user's medical history, fitness data, medical literature, geolocation data, and input from questionnaires.

10. The system of claim 8, wherein the processor is further configured to:
identify a plurality of diseases that the user is at risk of developing or has been diagnosed with;
generate a hierarchy of the diseases to identify a parent disease that contributes to one or more child diseases; and
select the behavioral change to mitigate the development of the parent disease.

11. The system of claim 8, wherein the processor is further configured to:
analyze, for a population of users, characteristics of electronic notifications provided to corresponding cohorts that successfully promoted a behavioral change in order to determine optimal characteristics of the electronic notifications; and
dynamically adapt characteristics of the electronic notifications provided to the cohort to the optimal characteristics in order to achieve a reduced response time of the user with regard to adopting the behavioral change.

12. The system of claim 8, wherein the processor is further configured to:
analyze, for a population of users, a composition of corresponding cohorts for the population of users that successfully promoted a behavioral change in order to determine an optimal cohort composition; and
dynamically adapt a composition of the cohort to the optimal cohort composition in order to achieve a reduced response time of the user with regard to adopting the behavioral change.

13. The system of claim 8, wherein the processor is further configured to:
determine that the behavioral change has not occurred within a specified timeframe; and
increase a frequency of notifications to the cohort until the behavioral change occurs.

14. The system of claim 8, wherein the processor is further configured to:
determine that the behavioral change has not occurred within a specified timeframe; and
modify the cohort by adding members that have been successful with promoting behavioral changes and/or removing members that have not been successful with promoting behavioral changes.

15. A computer program product for adaptively promoting a behavioral change, the computer program product comprising a computer readable storage medium having program instructions embodied therewith, the program instructions executable by a processor to:
analyze a medical history of a user to identify a risk of developing a disease;
determine a behavior of the user that promotes the development of the disease;
map the behavior to a behavioral change, wherein the behavioral change mitigates the development of the disease;
promote the behavioral change to the user through a cohort, by providing electronic notifications to the cohort; and
adaptively modify the electronic notifications provided to the cohort until the user adopts the behavioral change.

16. The computer program product of claim 15, wherein the program instructions executable by the processor further include instructions to:
identify a plurality of diseases that the user is at risk of developing or has been diagnosed with;
generate a hierarchy of the diseases to identify a parent disease that contributes to one or more child diseases; and
select the behavioral change to mitigate the development of the parent disease.

17. The computer program product of claim 15, wherein the program instructions executable by the processor further include instructions to:
analyze, for a population of users, characteristics of electronic notifications provided to corresponding cohorts that successfully promoted a behavioral change in order to determine optimal characteristics of the electronic notifications; and
dynamically adapt characteristics of the electronic notifications provided to the cohort to the optimal characteristics in order to achieve a reduced response time of the user with regard to adopting the behavioral change.

18. The computer program product of claim 15, wherein the program instructions executable by the processor further include instructions to:
analyze, for a population of users, a composition of corresponding cohorts for the population of users that successfully promoted a behavioral change in order to determine an optimal cohort composition; and
dynamically adapt a composition of the cohort to the optimal cohort composition in order to achieve a reduced response time of the user with regard to adopting the behavioral change.

19. The computer program product of claim 15, wherein the program instructions executable by the processor further include instructions to:
determine that the behavioral change has not occurred within a specified timeframe; and
increase a frequency of notifications to the cohort until the behavioral change occurs.

20. The computer program product of claim 15, wherein the program instructions executable by the processor further include instructions to:
determine that the behavioral change has not occurred within a specified timeframe; and
modify the cohort by adding members that have been successful with promoting behavioral changes and/or removing members that have not been successful with promoting behavioral changes.
